(19) United States
(12) Patent Application Publication
(10) Pub. No.: US 2021/0353384 A1
(43) Pub. Date: Nov. 18, 2021

(54) HANDPIECE, SYSTEM AND METHOD FOR DENTAL IMPLANT GUIDED SURGERY

(71) Applicant: Fei Gao, Buena Park, CA (US)

(72) Inventor: Fei Gao, Buena Park, CA (US)

(21) Appl. No.: 15/930,246

(22) Filed: May 12, 2020

(51) Int. Cl. A61C 1/08; A61C 1/10; A61C 8/00; A61C 9/00; A61B 6/14; A61B 6/03

(52) U.S. Cl. CPC A61C 1/084; A61C 1/10; A61B 6/032; A61C 9/0053; A61B 6/14; A61C 8/0089

(57) ABSTRACT

A guided handpiece, system, and method to perform image guided surgery for dental implants. Conventionally an image guided surgery system needs a surgical guide, a handpiece, a set of guided drills and drill adaptors. Extended drills must be specifically designed and made for implant brands and parameters. Adaptors are required to accommodate drills of different diameters. The process is much more complex than freehand surgery while surgical guides are not used. This invention introduces a guided handpiece, corresponding system, and method, which overcome the problems of prior art and make it possible to perform guided surgery with regular drilling tools.

### CROSS-REFERENCE TO RELATED APPLICATIONS

Not Applicable

### REFERENCE CITED

| | | |
|---|---|---|
| EP1968475B1 | 2007 | ESPOSTI, Alessio |
| U.S. Pat. No. 8,832,019 | 2011 | GAO, Fei |

### BACKGROUND

The major tools for image guided surgery of dental implants include a handpiece (drilling device), guided drills, and a surgical guide. FIG. 1 illustrates the basic approach. A drill 4 is inserted into a handpiece 6 and is driven into the patient's anatomy with the guidance of the suigical guide 8, which has so-called guiding holes or drilling sleeves 10. Tire steps to drill implant l oles and to place implants are very similar, except that surgeons use insertion tools or implant mounts in the placement step.

Although the idea of guided drilling is simple, the actual implementation has many different factors and parts. FIGS. 1(a) and 1(b) illustrates the drilling operation for a 5×10 mm implant. The surgical guide has been designed so that the top of the guide is 9 mm above the top of the final implant position, which is called prolongation value. The surgical guide has a hole bigger than the implant, say, 6 mm.

In order to drill through the hole, the current approach needs more than one drills. In minimum, we need 2×9, and 4.5×9 drills. In a typical scenario, the implants can be pressed into the 4.5 hole after the drilling in FIG. 1(b). Both drills have to have 9 mm prolongation over regular drills, i.e., 9 mm longer, so that they can match the height of the surgical guide. Such longer drills are called guided drills. For every single implant and every single regular drill for the implant, the manufacturer has to design and make guided drills which are longer by 9 mm. In order to facilitate the situations such as various gingiva thickness, tooth extraction, etc., different prolongations are required. As a result, in many guided surgical kits, the manufacturers offer multiple prolongation values. A regular drill for 10 mm implants now needs guided drills of 17 mm, 19 mm, 21 mm for prolongation values of 7 mm, 9 mm and 11 mm.

The guided drills very often make the guided surgery complex in many aspects. For example, with surgical guides designed with 9 mm prolongation value, drills are made with same amount of extension. If a drill has to be inserted into the guide from the top of the guide, an 18 mm total extra height of space inside the mouth is required in order to fit the surgical guide and handpiece. For many patients this is not practical at all. Special surgical guide designs and handling process will have to be implemented.

In addition, a drilling hole or a sleeve on a surgical guide has a set diameter, but osteotomy is performed step by step staring with 2 mm hole, then 3 mm, 4.5 mm, etc., until the final. We will need adaptors for the drills of different diameters so that they can fit into the guiding hole. In the example of FIG. 1, an adaptor 2 has a tube and a handle. The adaptor for 2 mm drill has 6 mm outer diameter, and 2 mm inner diameter. The adaptor for 4.5 mm drill has 6 mm outer diameter and 4.5 mm inner diameter. If surgeons want to drive the 5 mm implants into the implant site, surgeons need another adaptor of 5 mm inner diameter to ensure the accurate placement. Using surgical guide for both drilling and placement is called fully guided surgery. If the placement is not guided, or if some of the drilling steps are not guided, the final accuracy can be barely guaranteed.

The combination of all such drills and adaptors typically is called a guided surgical kit. Additional tools for a guided kit are helpful but not necessary, such as anchor pins, bone mills, cortical drills, tissue punches.

Many surgeons don't like the adaptors because adaptors need one hand to hold at surgery time. The surgical kit approach in FIG. 1 has been replaced by a new design as in FIG. 2, where each of the guided drills has a guiding cylinder 20. Corresponding to one drilling sleeve size, all the cylinders have same diameter as the hole of the sleeve. The drilling operations are illustrated in FIG. 3, where no adaptor is needed. The surgeon will just replace one drill with another to gradually finish the osteotomy from pilot drill to final drill. They may or may not have different prolongation values depending on the actual designs.

This approach eliminates the need of drilling adaptors but leads to another problem. For each diameter, drills of different lengths are required. FIG. 4 shows that a drill cannot be guided at the beginning because the drill length, if same as implant, will be longer than the drilling sleeve, and the guiding cylinder 20 is way above the drilling sleeve of the guide 8 when the drilling starts. Because of this, the surgical kit will have to provide drills of different length for a single length of implant, so the surgeons can start with a shorter drill with the guiding cylinder inside the sleeve.

In a nutshell, this approach is to mount an adaptor to the top of a drill, or to combine an adaptor with a drill.

In order to avoid using drilling adaptors, patent EP1968475 introduced a different paradigm that essentially mounts an adaptor (or guiding member as referred in EP1968475) to the drilling handpiece so that surgeons no longer need to hold the adaptor. That invention disclosed a dental handpiece comprising: a housing, a tool retaining mechanism for retaining a rotary dental tool, and a tubular guide member mounted coaxially with the rotary dental drill. The guide member has a tubular part for locating in a bore hole of a template for guiding the working path of the handpiece during use. The guiding part is not rotating with the drill. It is mounted below the tool retaining structure, on the cover plate, around the inlet to the head.

Mounting the adaptor onto the housing or body of the handpiece is basically same as mounting it onto a drill. It introduces same problems as the drills in FIG. 2 do. When an adaptor is a separated component in FIG. 1, drills move up and down inside the adaptor so the adaption can happen at any relative position among the drills, the adaptors and the metal sleeves on the surgical guide along the axial orientation. In other words, this diameter adaption is functioning at the tip, middle and upper part of the drill even though the adaptor is much shorter than the drill. However, when the adaptor is fixed on the bottom of the handpiece or top of the drill, it will not function before the drills go low enough or even below the soft tissue level.

EP1968475 introduces telescope like adaptor design or retractable guiding member to overcome the issue, and this makes the design very complex and not practical. There is no known implementation of this design in the industry. Instead the modified drill design as in FIG. 2 is becoming very popular.

Another essential problem behind EP1968475 is that the implant lengths are all different, drill lengths are not standard, the entire system including handpiece, the retractable guiding member and surgical template have to be configured so that they work properly with any implants. That disclosure cannot lead to a single simple scheme for such adjustments or configurations.

EP1968475 also does not overcome the issue of needing longer drills. It specifically said dedicated drills from manufacturers are needed. As a matter of fact, none of the treatment planning and design software systems including GuideMia, 3Shape, Nobel Clinician, Simplant, etc supports this approach.

The complexity of existing guided surgical kits does not stop here. Typically implants have taper angles, so do the corresponding drills. The extended drills must have the same kind of taper angles as regular drills if the surgeon wants to complete the drilling and implant placement with a surgical guide. When different manufacturers have different taper design for their drills, people must have one sets of drills for each implant design. As a matter of fact, there is no industry standard at all as far as the implant lengths, diameters and tapper angles are concerned. This makes it very hard and impractical for dentists to adopt different implant brands for the purpose of guided surgery.

In order to deal with this, the idea of universal surgical kit has been proposed. The idea is to use cylindrical drills with a surgical guide to prepare an implant site to certain size, and use the regular drills corresponding to the actual implant to finish drilling without surgical guides. The cylindrical drills have a set of chosen diameters regardless of the actual implant size, such as 2 mm, 2.5 mm, 3 mm, 3.5 mm, 4 mm, etc.

With the introduction of universal kit, dentists can perform guided drilling to the step before the final drills, but not the final. The final drill without surgical guide is illustrated in FIG. 1c. This is not a fully-guided approach, but is a very widely used one because the doctors do not have the burden to acquire guided surgical kits for each implant brands. In a quite common scenario, a 5.0 mm implant may have 2.5 mm diameter at its apex, thus the cylindrical drill used for guided drilling can only go up to 2.5 mm, then the surgeon has to take out the surgical guide, drill one or more times with regular drill to prepare the tapered shape to 5.0 mm at the entrance. For this scenario, guided drilling plays a very limited role in the process, and the final drilling and placement errors can be as big as freehand drilling.

Moreover, it is a great deal to change implant length during a surgery. With the surgical kits discussed above, especially universal surgical kits, when a surgeon wants to change implant length onsite, the total drilling length, the drill length, and prolongation values will all need to be reconciled, which is very sophisticated even for engineers, let alone in a clinical setting.

To summarize the industry state-of-art, current guided implant surgery is performed with guided drills and various combinations. Specially designed surgical drills are always needed, either as universal kits or made according to underlying implants. There is no fully guided solution for all implants, and there is no way to just use regular drills with surgical guides. This has been greatly hindering the application of guided surgery technologies.

With all those existing problems in mind, the objective of this invention is to perform fully guided implant osteotomy with surgical guides with only regular drills that are not extended or modified otherwise. The surgeons will be able to use the drills and handpieces with or without surgical guides in the same drilling protocol.

### BRIEF DESCRIPTION

This invention introduces a guided handpiece that can work with regular drills with the structure redesigned, and the entire guiding system is based on the handpiece, surgical guides, and accessories. The invention has the following major features:
1. The invention redesigns handpiece by adding a guiding part so that it can fit into the drilling sleeves on a surgical guide. When the guided osteotomy drilling is performed, the handpiece is being guided by the holes or sleeves.
2. The guiding part is attached to, or is the same component as, the drill retaining and clamping structure of the handpiece. This is a critical feature that eliminates the need for extended drills. The guiding part will have a hole for inserting and clamping the drills. The size of the hole is same as the upper shaft of drills, which is normally a standard size.
Alternatively, the guiding part and the clamping structure are two mechanical parts, but have to be nested and overlapping same area along the axis of the drill.
Either way, the clamping structure is moved outside of the head of the handpiece. The guiding part, the clamping structure, and the clamped shaft of the drills are substantially on the same position along their axis. They are not arranged as one next to another along the axis. In contrast, the clamping structures are always above the guiding parts or adaptors in prior art.
The lower end of the guiding part will be substantially at the same level of the gum level of a drill, that is, the transitional area or neck area between the clamping shaft and the drilling area.
Either as a separated component or same part as the clamping structure, an extra cylindrical tube can be attached outside of the guiding part as a diameter adaptor so as to work with different size of implants and drilling sleeves.
3. The guiding part further may have a stopper that can prevent drill from going excessively deep.
Preferably the stopper can be adjusted to a different height along the axis.
4. The guiding part works with regular drills with no prolongation. The drills do not need to be extended in any manner. The regular drills designed for conventional drilling without surgical guides are simply used, and the drilling sequences and protocols remain the same.
5. The outer diameter of the guiding part is bigger than the implant's final drills. For example, 4 mm can work for any implant below 4 mm. 4 mm and 5.5 mm are typically preferred dimensions that can fit most of the implants.
6. The surgical guide has metal sleeves that are specially designed to match the guiding part of the handpiece. The sleeves typically have cylindrical holes. The height of a sleeve together with gum thickness is substantially same as the length of the guiding part on the handpiece, and substantially same as the length of an implant. This arrangement will make sure the total guided drilling depth is about the same as the implant length, and the entire or most of the guiding part will go into a sleeve when a drilling operation is complete.
7. A depth drilling adaptor is part of the drilling system. The adaptor is used as an extension to the sleeves, along the axis of the sleeves, not as a diameter adaptor. When a drilling operation starts, the drill is inside the sleeve, but the handpiece guiding part is still outside of it, so an adaptor is used to extend the sleeve.
8. A computer software module will be needed to determine the actual height of the surgical guides, stopping positions of the sleeves, height of the sleeves, etc. Since not all implant drills have same shank length, the software module will be used to adjust the surgical guide design so that the difference of the shank lengths will be compensated.

Ever since the introduction of guided surgery, one of the things the industry is trying to look into is the prolongation of guided drills, and to come up with a solution to reduce the handpiece form factor. Unfortunately, there is barely any room for the engineers to improve this because having an appropriate height is important to maintain good drilling accuracy. With this invention, the total form factor of the handpiece together with drills is not changed, but the guidance provided by the handpiece is much more improved than the prior art.

Throughout this disclosure, tissue thickness will play an important role in explaining the drilling process and the parameters of the components. The term tissue thickness unless otherwise explained should be interpreted as the combination of tissue thickness at an implant site together with the distance the implant will be placed below the tissue.

It is important to point out that in this invention, the guided operation must meet the following criteria:

1. allowing different sizes of the handpiece guiding parts,

2. allowing the up and down sliding movements of the handpiece guiding part inside the drilling sleeves, and

3. allowing the rotation of the handpiece when the guiding part is inside the sleeves, for which the guiding part should share the same axis as the drill.

It is naturally understood that item 1 and 2 are necessary. The third item means when the handpiece is inside the drilling sleeves, we should allow the dentists to rotate the handpiece for convenience purpose or to avoid interfering with adjacent teeth. In other words, any designs that require fixed angular position of the handpiece is not same as this invention. For example, one can move the guiding part to be away from the drill axis. This way, the underlying drill is fixed at the implant axis, the guiding part on another axis, so the handpiece cannot rotate around the drill axis.

### DRAWINGS

FIG. 1 illustrates a typical surgical kit configuration and drilling protocol. A surgical guide is designed according to the treatment plan. Guided drills and adaptors are used for drilling operations from pilot to final.

FIG. 2 shows modified surgical drill design, which combines a conventional drill and a guiding part.

FIG. 3 shows the drilling steps using the drills with guiding part.

FIG. 4 illustrates the problem that drills are not guided at the beginning of a drilling operation, so shorter drills must be used at the first step.

FIG. 5 is the overall design of the guided handpiece and how it holds a drill, where the drill clamping part and the guiding part are a same component. Alternatively, they can be two parts but installed together. Both are below the head of the handpiece.

FIG. 6 shows the guided handpiece holding the implant drill slides into a drilling sleeve of a surgical guide, with the guiding part of the handpiece guide by an adaptor 46 with a handle 50.

FIG. 7 shows the idea of long drilling sleeves with a flange in the middle and one end outside of the surgical guide.

FIG. 8 shows that stoppers can be further added to the sleeves so they can couple with the stoppers on the guided handpiece.

FIG. 9 shows diameter adaptors of different sizes that can make the guiding part work with different drilling sleeves and hence different implants.

FIG. 10 shows how errors can happen with drilling operations due to the clearance between drilling sleeves and drills or adaptors.

FIG. 11 further illustrates the design of a depth adaptor that guides handpiece into the surgical guide. The adaptor has same inner diameter as the guiding part of the handpiece and the drilling sleeve. With a C-shaped open design, the adaptor can slide in and out laterally during a drilling operation. It has a thinner section on the lower end that can be inserted in a drilling sleeve as in FIG. 12. The sleeve has a groove to receive the adaptor.

### DETAILED DESCRIPTIONS

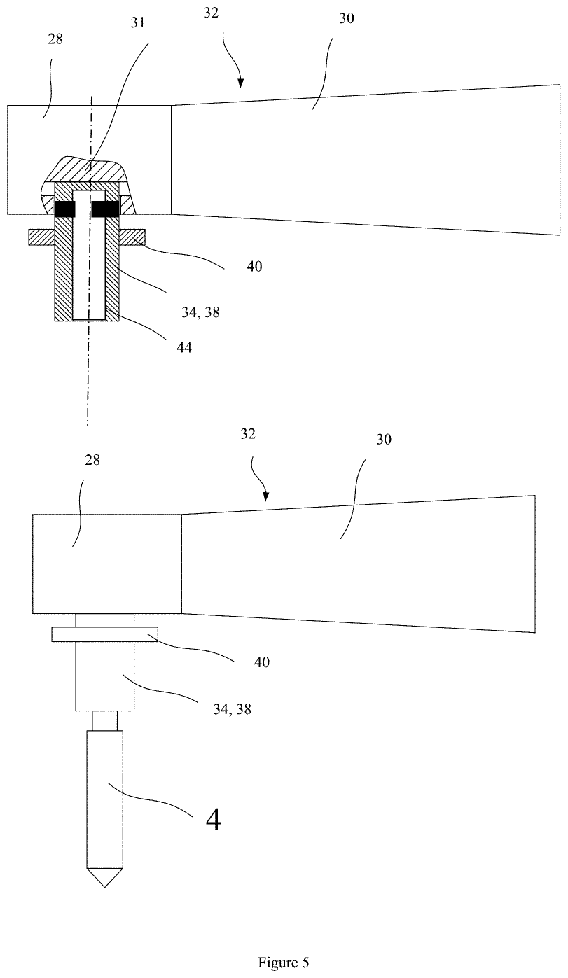

FIG. 5a shows modified handpiece design and FIG. 5b the approach to guided surgery with a guiding part on the handpiece. Handpiece 32 has an extended guiding part 38 in its housing component 31. The guiding part goes into the drilling sleeves on the surgical guide. Drill 4 in this disclosure should be understood as an example of rotary dental tool, or collectively, implant drills, implant drivers, implant mounts, tissue punchers, bone profilers, etc. Drilling or drilling operations should be understood as any operations performed by said dental tools.

With conventional handpiece design, on the upper part of the drill, there is a clamping mechanism to hold the drill in the top position. The modification this invention makes is to move the clamping structure 34 lower and preferably combine it with the guiding part 38.

For the sake of convenience, we use Z axis to represent the axis of the drill, or the rotation axis of the rotary part. The clamping component serves as the guiding part too. It is extended along Z axis below the head to the working part of the drills, and its inlet for drills are moved lower. This guiding part or extended clamping structure is cylindrical.

The most important feature of this invention is that the guiding part, the drill clamping structure, and the shank area of a drill overlap each other along the Z axis, or, they are substantially at the same position along Z axis. On the other hand, with all prior art, the guiding area is below the clamping area, either with the drill adaptors, with the drills with guiding area, or with the modified handpiece of EP1968475B1.

At the top of the guiding part is stopper 40. This stopper is meant to stop the drills or handpiece from going excessively deep. Depending on actual parameters of different components, the stopper can be on the guiding part, or the body cover plate at the bottom of the head 28. Additional adjustment mechanisms such as threads or pins can be added to the actual structure so that the stopper can be moved along the axial direction of said guiding part.

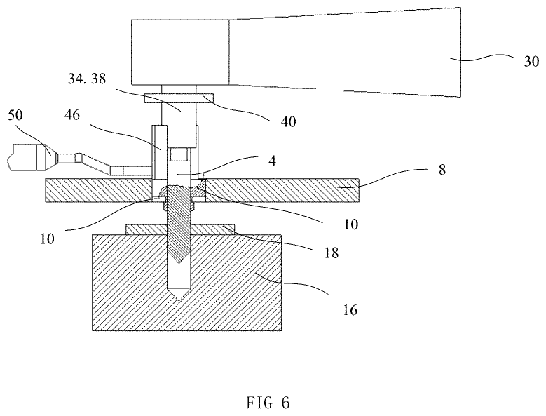

As FIG. 6 indicated, the distance the drill tip goes into the patient anatomy should be the length of the guiding part if we want the drilling is completely guided. Given that, the length of a guiding part should be the sum of the length of an implant and the soft tissue thickness. A shorter guiding part cannot provide complete guidance, and longer one on the other hand must have a stopper. The length of the clamped shaft of a drill is about 14-15 mm. Given a regular implant of 12 mm long, typical tissue thickness of 3 mm, so the guiding part is also roughly in the 15 mm range as the clamping structure is. This is why it is a critical feature that the clamping structure and the guiding part is a single mechanical part or, if not, they are substantially on the same Z axis position.

The implant surgical guide and the sleeves are specifically designed for the handpiece. For an implant site, the combination of the tissue thickness, clearance between guide and tissue, and the total height of the sleeve is called guiding length. As illustrated in FIG. 6, the guiding length goes from the top of the sleeve 10 to the bottom of the gum 18. In the remainder of this disclosure, when accurate numbers are not concerned, sleeve height and guiding length can be understood as the same. With prior art, this is typically referred as prolongation value, the difference between an implant length and its guided drill, or difference between a regular drill and guided drill.

Alternatively, the guiding part and drill clamping structure can be two co-axial tubular design. The clamping structure is attached to the handpiece housing. The guiding part can be either attached to the clamping structure or mounted on the handpiece head. With this embodiment, the guiding part can be interchangeable. A series of guiding parts with different diameters can be provided and the surgeons can replace a guiding part with another to accommodate the needs of different implant or drill diameters.

The guiding length is correlated to the total length of the guiding part of the handpiece. In the maximum, the total sleeve height is same as the total length of the guiding part but cannot be more than that.

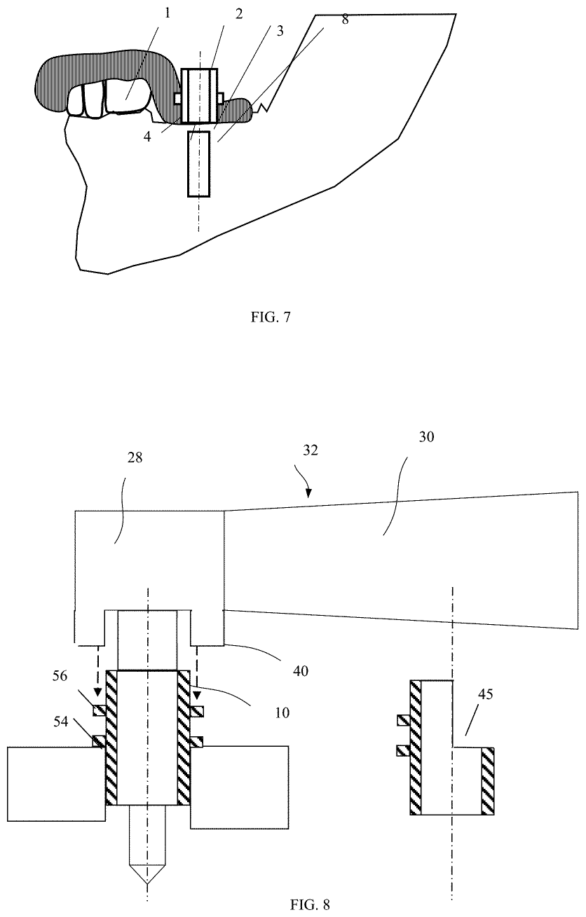

As the guiding length is roughly the implant length, it is not necessary to make surgical guides of the same height. The guides can be shorter, and a drilling sleeve is divided into two sections, with one inside the surgical guide, one above. This is shown in FIG. 7. In a preferred design, a flange is added to the sleeve between the two sections so that the surgical guide provider can easily install the sleeves into the surgical guide with the flange as a stopper.

When a drilling operation is being performed, the surgeons need to know when to stop the drilling. In the simplest embodiment, when the stopper on the handpiece reaches the top of the drilling sleeve, the operation should stop.

In order to further control this, various stopper designs can be derived from this approach. In FIG. 8, an alternative is illustrated. The stopper 40 on the guiding part is relocated to the body of the handpiece, and stopper 56 is added to the drilling sleeve 10. At the time of actual drilling, the guiding part of the handpiece goes into the sleeve, and then stopper 40 reaches stopper 56 on the sleeve, the drilling should be stopped. At that time, the tip of the drill will reach the desired apical center of the implant. In other words, the stopper on the sleeve actually provides a mechanism to adjust the total length of the guided drilling.

The approach to add a stopper on the handpiece body leads to more flexibility of the guiding length variations. When the stopper is on the guiding part, the only condition to stop drilling is when the stopper reaches the top of the sleeve. Now with FIG. 8 one can adjust the stopping length by adjusting the stopper on the sleeve.

In a preferred embodiment, markers are inscribed on the sleeves to indicate different implant length. The surgeons will simply adjust the stopper to a marker corresponding to an implant length. When the surgeons determine to change implant lengths onsite, they can simply adjust the stopper. Accordingly, in the surgical planning software a procedure is provided with all the structures and parameters to determine the stopper position.

Sometimes it is not possible to insert a drill into a sleeve from the top of the sleeve because the patient mouth may not be wide open. This happens often in the posterior area. A lateral opening 45 in FIG. 8 is added to the sleeve on the upper section so that drills can slide into the sleeve laterally. The width of the opening is bigger than the drill diameter and smaller than the inner diameter of the sleeve. Therefore, when the drill tip is at the lower edge of the opening area, from the top of the drill to the tissue level is the total height of the space that is needed to accommodate the drills. From the tip of the drill to the tissue level is about same as the corresponding dimension in a conventional surgical guide in prior art, which is the extra space that is needed with the introduction of surgical guide. In other words, even though the drilling sleeves are extended above the surgical guide, the total depth required to insert the drills remains the same.

Considering the guiding part of the handpiece can go in all the way to the tissue level, the actual height of the handpiece outside of the surgical guide is just the head part 28 as in FIG. 5, same as regular handpiece used in surgery with surgical guides. Therefore, this invention increases the guiding length without increasing the handpiece height. This is a major benefit of this invention.

Ideally, the guiding depth of the handpiece, the sleeve height, as well as the drill length should work in harmony, but there are situations when the guiding part is too short or too small, the sleeve is too low, etc. In those situations, additional parts or design changes will be needed.

As examples, when a patient has very thick tissue, or the operation is for an immediate extraction case where the surgical guide sleeve can be way above the tissue level, or the implant is placed way below the bone surface, or the desired drill is excessively long, an adaptor to extend the guiding length is introduced.

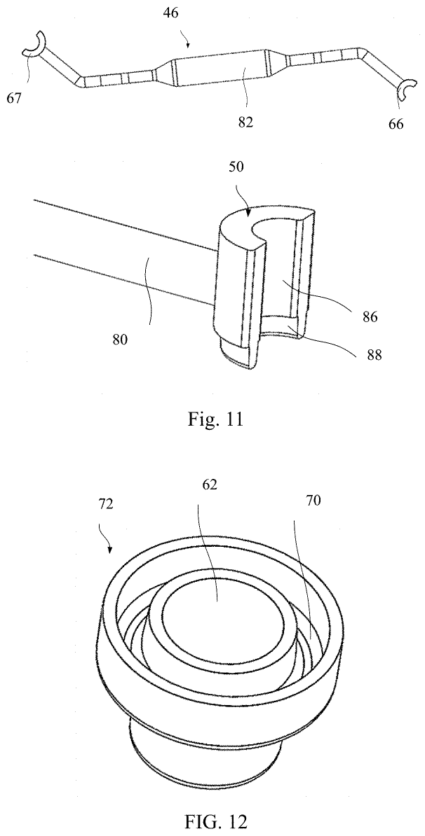

FIG. 11 shows a preferred embodiment to extend the sleeve with an adaptor that sits on the sleeve as in FIG. 6 and maintain same inner diameter as the sleeve. The adaptor sits on the sleeve and has a lower part 88 matching the outer diameter of the sleeve. In order for the sleeve to accommodate the adaptor part 88, the sleeve is modified as in FIG. 12, while the top of the sleeve will have a groove to receive 88. Its inner diameter is also the outer diameter of the guiding part of the handpiece. This adaptor further may have arm 80 and handle 50 for easy manipulation. In this embodiment, the adaptor is not a full cylindrical shape but a C shape so it can be taken out during the drilling process when the drill is inside. Adaptors can be attached to both ends of the handle. In FIG. 11, both ends are rotated by 45 degrees clockwise toward the same direction. Instead, arm 67 can rotate in opposite direction so that 66 will be used in the patient left side, 67 in the right side, or other way around.

In FIG. 5, when the drill just gets into the patient's anatomy, the guiding part is above the drilling sleeve, the surgeon can insert this adaptor between the guiding part of the handpiece and the drilling sleeve so that the drilling part can be guided into the drilling sleeve, then the adaptor can be removed laterally with its C shaped design.

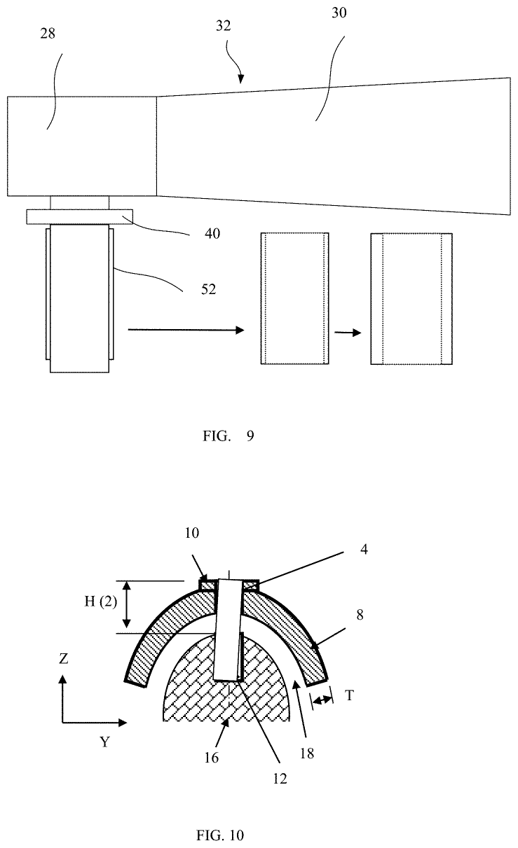

The diameter of the guiding part can be determined by the drill and implant diameters. In order to guide implants into the operation site, the inner diameter of the sleeve should be no less than the implant diameter and be same as the guiding part of the handpiece. For the handpiece, one would prefer to have same diameter so that the surgeons would not need to replace the handpiece during the surgery from one implant to another. However, it is not preferred to use same sleeve diameters on the surgical guides, because the available space can be very limited on the anterior site, while it can fit in a bigger sleeve in molar area. The diameter adaptor is introduced as illustrated in FIG. 9. In an embodiment, the guiding part of the handpiece can have one small diameter that can fit for anterior area, and work with the adaptors to cope with larger sleeve and implants in the posterior area.

One of the major technical issues with guided surgery and the guiding tools, whether guided drills or guided handpiece, is the accuracy of the osteotomy and implant placement. Until one actually looks into the accuracy issues, various guided surgery solutions look quite similar, but the subtle differences in the design in this invention leads to substantial differences in the accuracy of surgical outcome.

With the extended length of the drilling sleeve, the total guided drilling length is longer than prior surgical guide approach. With a conventional surgical guide, and also with EP1968475, the total guiding length is the sleeve height. As shown in FIG. 8, with a typical configuration, assuming the prolongation value is 9 mm, tissue thickness is 3, and a clearance is introduced between the guide and the tissue is 1 mm, then the guided drilling length is 5 mm. With this disclosure, the sleeve is extended upward to almost the length of the underlying implant. Mechanically, in order for a drill or adaptor to move freely inside the sleeve, there needs to be a clearance between them. In U.S. Pat. No. 8,832,019 B2, the applicant analyzed the error factors and provided an error simulation system. When the sleeve length is increased, the accuracy will be much more improved.

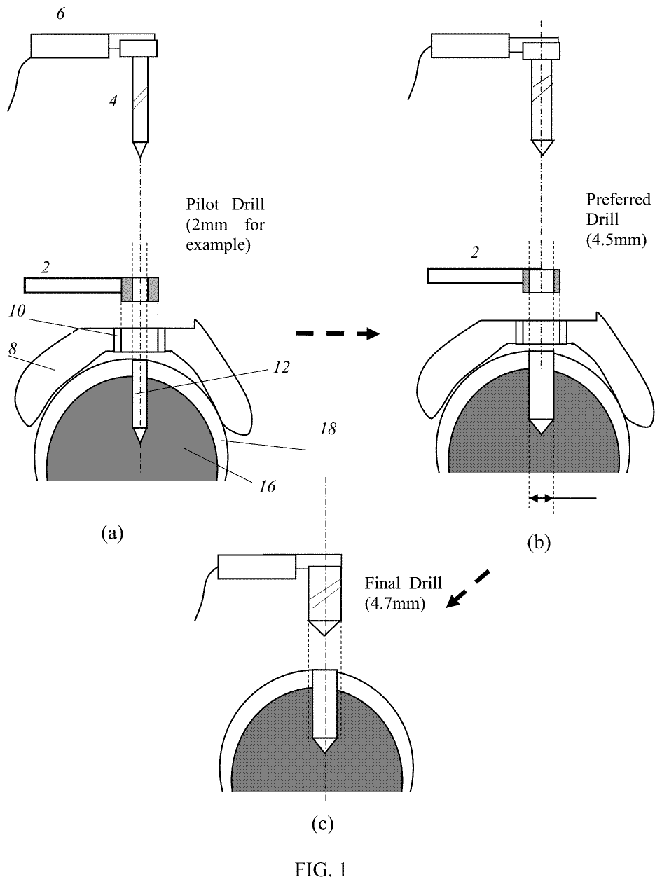

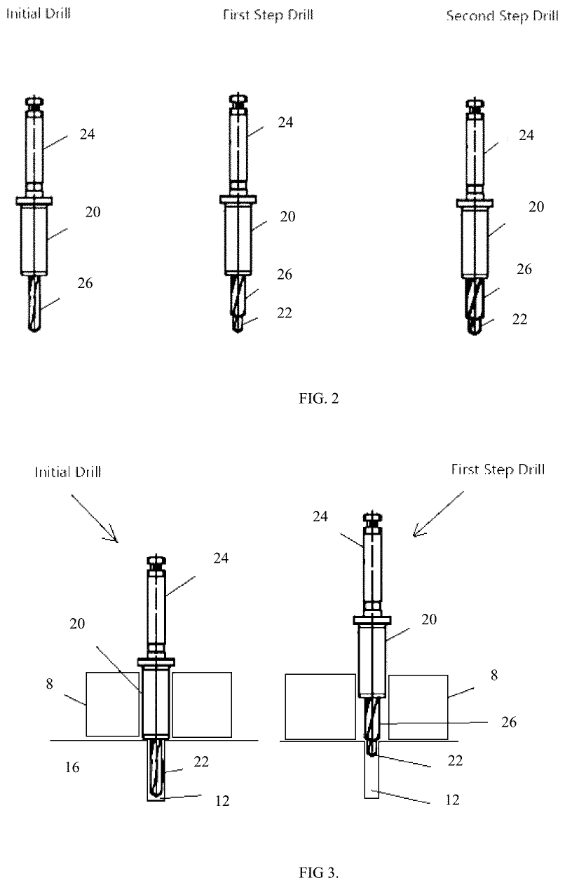

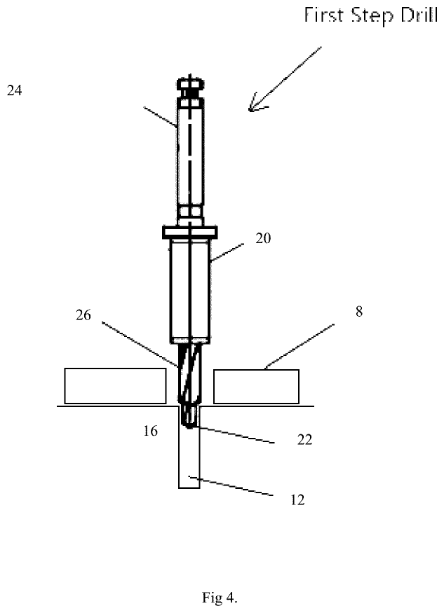

As in FIG. 10, assuming the clearance between the sleeve and an adaptor or drill is 0.1 mm, the sleeve height is 5 mm, prolongation is 9 mm, and implant length is 15 mm. The center axis can deviate by 0.2 mm (double the radius clearance) at lower edge of sleeve 4 (over 5 mm Z axis span), this can lead to 4.8 (((15+9)/5) times difference or 0.96 mm at the apical center of the implant. With prior art, when an adaptor is involved as in FIG. 1, assuming 0.1 mm gap between the adaptor and the drill, and 0.1 mm between the adaptor and sleeve, the final error will be doubled at 1.92 mm with extreme error situation, even with an assumption that no other handling problems will contribute to the error. This error is beyond a commonly accepted safety zone of 1.5 mm.

With this invention, the total sleeve height is increased to about 15 mm, this error at the apical center of the implant is reduced to about 0.4 mm. With the design that the guiding part and drill clamping part is the single component, there is no second gap, so the final accuracy is much higher.

As far as the accuracy is concerned, the characteristics of this invention is summarized as:
1. The total guiding length of the handpiece, with optionally an adaptor, is substantially same as the distance between the apical center of the desired implant position and the tissue surface.
2. The total height of the sleeves on the surgical guide is substantially same as the guiding length of the handpiece, with optionally an adaptor.
3. The guiding part of the handpiece is attached to the implant clamping structure so that the axial error between the two is substantially eliminated.

With the guided handpiece and accessories, a guided surgery system for dental implant drilling and placement now includes the following essential components:
1. A guided handpiece with guiding part and drill clamping part at the same Z axis level. In an alternative design, the guiding part is a separate component, and the system can have a set of guiding parts of different diameters. Whether they are same component or different, they are both below the head of the handpiece.
2. A set of drills from any implant manufacturer.
3. A surgical guide with drill sleeves, where the sleeves have heights substantially same as the guiding part of the guided handpiece.

The system further comprises accessories:
1. A series of diameter adaptors if only one guiding part is provided
2. One or more depths adaptor that can guide the guiding part of the handpiece before it can reach the drilling sleeves
3. Additional features of the system include but are not limited to:
   a) Stopper features on the guiding part or the handpiece,
   b) Stopper features on the drilling sleeves,
   c) Other tools in a guided surgical kit, such as trephine drills, cortical drills, bone mills, tissue punches, etc.

With the presented invention, the concept of “fully guided surgery” is now realized in a better way: all the osteotomy operations are performed with the surgical guide in place, all the implants are driven into the prepared hole with the surgical guide in place.

In prior art the drilling sequence of guided surgery depends on whether the guided drills are specifically made for the underlying implants. If not, and usually not, a universal surgical kit is used and the drills are not designed for the shape of the implant, so the process will be: place on surgical guide, perform pilot drilling using guided drills, perform additional drillings using bigger drills up to the point that the holes are slightly smaller than the apical diameter of the implant, take out surgical guide, use the conventional drills coming with implants to complete the final drill, and finally place implants without surgical guides. As described, it is not possible to perform fully guided surgery with this process.

With the present invention, fully guided surgery is possible for all implant systems by just using afore-mentioned drilling system and the conventional drills coming with the implants. Compared to conventional freehand implant osteotomy, this method has the same protocol to choose drill diameters, lengths, number of steps to drill, implant mounts or insertion tools. It is a very important benefit of this invention that no modification to drills is required.

With present invention, the method to perform guided surgery is summarized as below:
Step 1 perform dental implant treatment planning with patient CT data and/or model scans, simulate implant placement, design, and manufacture one or more surgical guides according to the parameters of the guided handpiece
Step 2 choose implant drills
Step 3 choose said guided handpiece and its accessories including depth adaptor, diameter adaptor, etc., install drills
Step 4 Use said guided handpiece to drill implant holes with the guidance of said surgical guides

The method also has additional steps after step 3:
Step 31 adjusting the stopper position on said guiding part of the guided handpiece in order to ensure the total drilling depth
Step 32 adjusting stopper position on the drilling sleeve if the adaptor on the handpiece is relocated from the guiding part to the head of the handpiece
Step 33 placing the depth adaptor over the drilling sleeves so that it can guide the drilling when said guiding part is still outside of the sleeves

What is claimed is:

1. a guided handpiece for dental implants osteotomy and placement, comprising
i) a rotary component driven by the mechanical system,
ii) a guiding part, which is installed co-axially with the rotary component,
iii) a drill clamping structure that can clamp and retain dental implant drills,
iv) said guiding part and clamping component are both below the head of the handpiece and substantially overlapping the same area along their axis,
v) one end of the guiding part goes into the drilling sleeves or guiding holes of a surgical guide and can move up and down in said sleeves or holes.

2. The said guided handpiece of claim 1, wherein said guiding part and housing component belong to a single part together so that they rotate co-axially.

3. the guided handpiece of claim 1, comprising a stopper that prevents said guiding part from going into said sleeves or holes of a surgical guide excessively deep by a distance more than predetermined.

4. the guided handpiece of claim 3, wherein said stopper can be adjusted and moved along the axial direction of said guiding part.

5. The said guided handpiece of claim 1, wherein said guiding part and drill clamping component are two separate components assembled together so that they rotate together.

6. the said guided handpiece of claim 1, wherein said guiding part and clamping component are two separate components, said guiding part is installed on the body of the handpiece, and the clamping component is installed on said rotary component.

7. the said guided handpiece of claim 4-6, wherein said guiding part is replaceable with designs of different outer diameters.

8. the said guided handpiece of claims 1 to 7, wherein the lower end of the guiding part is substantially at the same level of the soft tissue level of implant drills, that is, the lower end of the clamping shaft of the drills.

9. the guided handpiece of claim 1, wherein said guiding part has smaller diameter than the diameters of the sleeves or holes of the surgical guide, a diameter adaptor is mounted on the guiding part to fit into the sleeves or holes of the surgical guide.

10. the guided handpiece of claim 1, wherein said guiding part has smaller diameter than the diameters of the sleeves or holes of the surgical guide, a diameter adaptor is placed outside of the guiding part and mounted on the head of the handpiece in order to fit into the sleeves or holes of the surgical guide.

11. the guided handpiece of claim 1, comprising a depth adaptor as an accessory, which can guide the guiding part to into the drilling sleeves or holes and thus allows complete the drilling to the depth.

12. the guided handpiece of claim 11, wherein said length adaptor is a separate part of a non-closed C-shape design so that said adaptor can be taken out during a drilling operation and the surgery operator does not have to stop the drilling and pull out the drill from the surgical guide.

13. the system for dental implant osteotomy and placement comprising
a guided handpiece of either one of the claim 1-12, with or without said diameter adaptor and/or said depth adaptor,
dental implant drills or insertion tools, and
at least one dental implant surgical guide.

14. the system for dental implant osteotomy and placement of claim 13, wherein the surgical guide comprises, at least one drilling sleeves.

15. the system for dental implant osteotomy and placement of claim 14, wherein the height of said drilling sleeves is substantially same as the length of a dental implant or the working part of an implant drill so that the entire drilling course for an implant can be completely guided by the sleeve.

16. the system for dental implant osteotomy and placement of claim 15, wherein the combined height of said guiding part of said guided piece and said depth adaptor is substantially same as the height of drilling sleeve.

17. the method to perform dental implant osteotomy using said drilling system of claim 13-16 comprising the following steps:
Step 1 perform dental implant treatment planning with patient CT data and/or model scans, simulate implant placement, design and manufacture one or more surgical guides according to the guided handpiece
Step 2 choose and install the corresponding implant drills
Step 3 choose said guided handpiece and its accessories including depth adaptor, diameter adaptor, etc.
Step 4 choose said guided handpiece to drill implant holes with the guidance of said surgical guides

18. the method to perform dental implant osteotomy of claim 17, comprising additional steps after step 3:
Step 31 adjust the stopper positions on said guiding part of the guided handpiece in order to ensure the total drilling depth,
Step 32 placing the depth adaptor over the drilling sleeves so that it can guide the drilling when said guiding part is still outside of the sleeves.
